(19) United States
(12) Patent Application Publication
(10) Pub. No.: US 2004/0106927 A1
(43) Pub. Date: Jun. 3, 2004

(54) Vertebral distractor

(76) Inventors: Brian M. Ruffner, Metuchen, NJ (US); Erik O. Martz, Jackson, NJ (US)

Correspondence Address: Peter G Dilworth, Dilworth & Barrese, 333 Earle Ovington Boulevard, Uniondale, NY, 11553, US

(21) Appl. No.: 10/469,338

(22) PCT Filed: Mar. 1, 2002
(86) PCT No.: PCT/US02/06198

(51) Int. Cl. A61B 17/56

(52) U.S. Cl. 606/90; 606/61; 606/85

(57) ABSTRACT

An intervertebral distractor possesses a scissors-like mechanism for actuating its distal portion for the spreading of adjacent vertebrae. The distal portion of the distractor possesses a pair of extension members laterally offset therefrom with each extension member possessing a vertebral endplate-engaging member.

### CROSS-REFERENCE TO RELATED APPLICATION

This application claims the benefit of U.S. Provisional Application No. 60/273,307, filed Mar. 2, 2001, the entire contents of which are incorporated by reference herein.

### BACKGROUND OF THE INVENTION

1. Field of the Invention

This invention relates to an instrument and method for spreading apart adjacent vertebrae of a vertebral column to facilitate insertion of an implant into the intervertebral space. More particularly, the present invention relates to a distractor for the distraction of vertebrae using an anterior or posterior approach.

2. Background of the Invention

Back pain can be caused by either one or a combination of the following: a loss of disc height, compression of nerve roots, degenerative disc disease, spondylolisthesis, and other causes. The current standard of treatment for people suffering from severe back pain requiring surgical intervention due to different types of pathology is by intervertebral fusion. Intervertebral fusion is achieved by fusing two adjacent vertebral bodies together by removing the affected disc and inserting a suitably sized implant into the disc space that allows for bone to grow between the two vertebral bodies bridging the gap left by the disc removal.

Known intervertebral fusion procedures typically involve the steps of removing a portion or all of the affected disc material, spreading apart adjacent vertebrae with a distractor, and inserting an implant, e.g., allograft rings, c-sections, dowels, etc., or threaded cages, into the intervertebral space previously occupied by the removed disc material. This procedure can be done either from the front of the patient (anterior interbody fusion) or from the back (posterior interbody fusion).

### SUMMARY OF THE INVENTION

In accordance with the present invention, a vertebral distractor is provided which permits an implant to be inserted into the intervertebral space during distraction of adjacent vertebrae. The implant is inserted into the disc space between the vertebrae-spreading members of the distractor while the latter are in spreading contact with the opposed upper and lower vertebral endplates of the distracted adjacent vertebrae.

In accordance with the present invention, a vertebral extractor is provided which comprises:

a) a pair of operating members each of which possesses a proximal portion adapted for manual gripping and a distal portion adapted for spreading vertebrae, the operating members being connected to each other by a pivot member disposed between their proximal and distal portions;

b) a pair of extension members, each extension member being connected to the distal portion of each operating member and laterally offset on the same thereof; and,

c) at least one vertebral end plate-engaging member connected to each extension member.

During use of the intervertebral distractor to achieve extraction of adjacent vertebrae, the offset arrangement of its extension members with their corresponding vertebral endplate-engaging members defines a frame-like opening or aperture through which a fusion implant can be readily inserted into the intervertebral space without hindrance or obstruction.

In one embodiment of the foregoing distractor, each operating member of the distractor is made up of several articulating members possessing a sliding and pivoting action which permits parallel distraction of adjacent vertebrae.

Further in accordance with the present invention, an instrument set for carrying out an intervertebral fusion procedure is provided which includes the vertebral distractor of this invention and one or more other instruments employed in such procedure, e.g., one or more rasps for removal of tissue from a vertebral endplate, one or more trials, a fusion implant inserter, a slaphammer and a bone tamp.

### BRIEF DESCRIPTION OF THE DRAWINGS

FIGS. 1 and 2 are perspective views of a distractor of this invention in, respectively, the closed and the open (i.e., vertebrae spreading) positions;

FIGS. 3 and 4 are partial views of the distal portions of the distractor of FIG. 1 in, respectively, the closed and open positions;

FIGS. 5-7 illustrate the operation of the vertebral distractor of FIG. 1 to achieve the spreading of adjacent vertebrae in a distraction procedure; and,

FIGS. 8-12 show various views of a rasp which is especially well suited for use with the distracter of FIG. 1 in carrying out an intervertebral fusion procedure.

### DETAILED DESCRIPTION OF THE INVENTION

Preferred embodiments of the presently disclosed vertebral distractor of the invention will now be described in detail with reference to the drawings in which like reference numerals designate identical or corresponding elements in each of the several views.

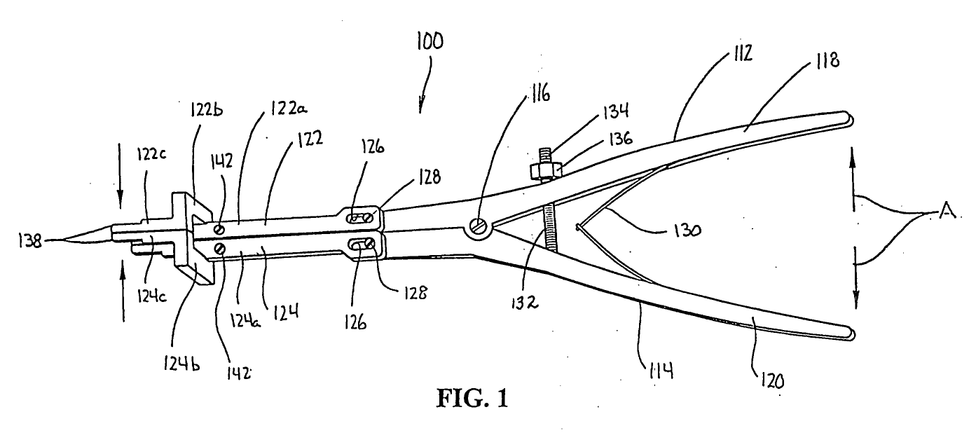

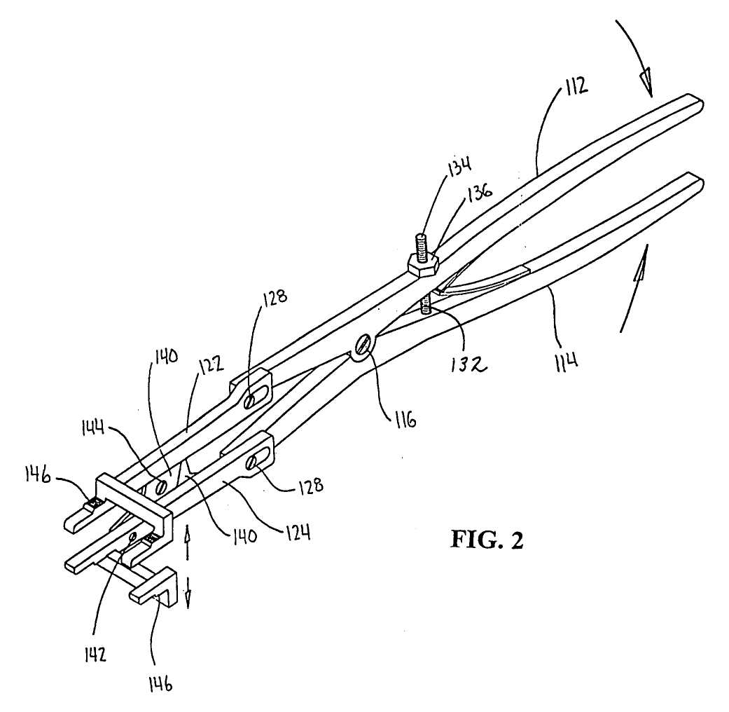

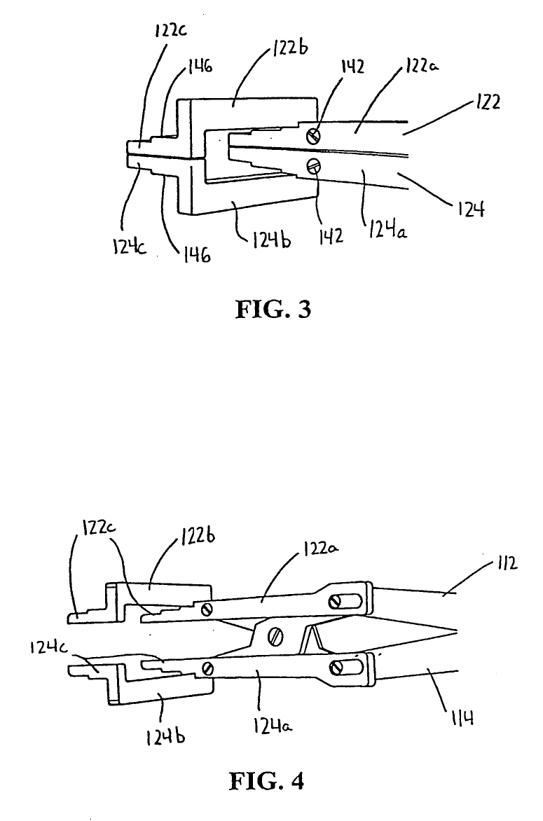

FIGS. 1-4 illustrate one preferred embodiment of the presently disclosed vertebral distractor shown generally as 100. Vertebral distractor 100 includes first and second operating members 112 and 114 which are pivotably connected to each other about a pivot member 116. Operating members 112 and 114 include grips 118 and 120, respectively, and jaws 122 and 124. Jaw 122 is secured to the distal end of operating member 112 and jaw 124 is secured to the distal end of operating member 114. A slot 126 is formed in the proximal end of each jaw. Slot 126 is dimensioned to slidably receive a pin which can take the form of screw or rivet 128. Screws or rivets 128 secure jaws and 122 and 124 to the distal end of operating members 112 and 114, respectively. A biasing member 130 is positioned between operating members 112 and 114 to urge grips 118 and 120 apart in the direction indicated by arrows “A” in FIG. 1. A locking member 132, including a screw 134 and nut 136, extends through an opening (not shown) in operating member 112 and is rotatably fastened to operating member 114. Nut 136 is threaded onto screw 134 to lock grips 118 and 120 at any position between open and closed positions of the jaws.

Each jaw 122 and 124 includes elongated body portions 122a and 124a, lateral extensions 122b and 124b, and a pair of spaced-apart arms 122c and 124c. If desired, the angle of spaced-apart arms 122c and 124c relative to the horizontal can deviate a few degrees therefrom so as to provide a lordotic angle or a kyphotic angle. Extensions 122b and 124b are laterally offset from the same side of jaws 122 and 124, e.g., at an angle of approximately 90° relative to the longitudinal axis of distractor 100, and in the open position, form a frame-like opening with unobstructed access to the intervertebral space thereby facilitating insertion of the fusion implant. The distal end of each arm has a smooth rounded surface 138 or other configuration, e.g., that of a wedge, to facilitate entry of arms 122c and 124c into the intervertebral space. Each of arms 122c and 124c includes a vertebral endplate-engaging 146 which engages a vertebral endplate after the arms have been within the intervertebral space. Vertebral endplate-engaging surfaces 146 are advantageously textured, e.g., grooved, to improve their gripping contact with the vertebral endplates.

A pair of support members 140 are secured to a side of jaws 122 and 124 opposite lateral extensions 122b and 124b. The distal end of each support member 140 is pivotally fastened to the one of jaws 122 and 124 by a screw or rivet 142. Support members 140 are rotatably connected to each other by rotating member 144 and are held in close alignment with jaws 122 and 124 to support, or stabilize, the jaws during movement of the jaws between approximated and distracted positions.

The various elements constituting vertebral distractor 100 are aligned along a common longitudinal axis. However, if desired, the distractor can feature one or more bends or curves in a direction which is opposite the direction of extensions 122b and 124b so as to place the distal portion of the instrument at an angle to the proximal portion of the instrument. Thus, e.g., a bend or curve of a few degrees can be introduced into the instrument between pivot member 116 and jaws 122 and 124 and/or at the distal portions of jaws 122 and 124, e.g., at about the location where extension members 122b and 124b are connected to the jaws. This arrangement provides even greater facility of insertion of the fusion implant into the intervertebral space since it displaces the proximal portion of the instrument away from its vertebral endplate engaging members.

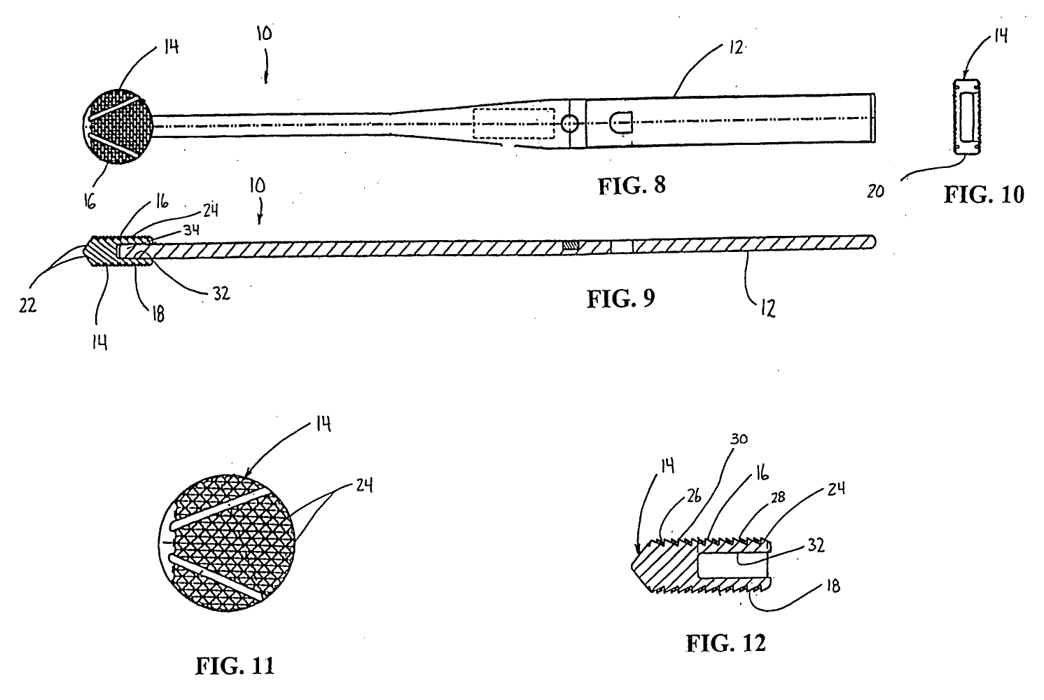

FIGS. 8-12 illustrate a preferred type of rasp, shown generally as 10, which can advantageously be used in conjunction with the vertebral distractor 100 of this invention in an intervertebral fusion procedure. Rasp 10 includes an elongated handle portion 12 and a head portion 14. Head portion 14 has a circular configuration and includes upper and lower surfaces 16 and 18 and a sidewall 20. Alternately, head portion 14 may assume other configurations including triangular, rectangular, square, etc. The distal end of 22 of upper and lower surfaces 16 and 18 is tapered to facilitate entry into the intervertebral space. Each of the upper and lower surfaces 16 and 18 includes a multiplicity of protrusions 24 formed thereon. Protrusions 24 have a triangular configuration including a preferably, substantially arcuate wall 26, a sloped wall 28 and an edge 30. Alternatively, wall 26 maybe substantially flat and vertical. The configuration of protrusions 24 will effect abrasion only when rasp 10 is moved over a surface in the forward direction. When the rasp is moved in a rearward direction the sloped wall will slide over the surface and no abrasion to a surface will occur. Alternately, the orientation of the protrusions may be altered to provide abrasion during rearward movement of head portion 14 or during forward and rearward movement of head portion 14. Further, abrasion may be provided by rotational movement of head portion 14 about the longitudinal axis of the spine.

As illustrated in FIGS. 9 and 11, head portion 14 includes a bore 32 dimensioned to receive the distal end 34 of handle portion 12. Head portion 14 and handle portion 12 may be permanently attached using any known fastening technique including welding, brazing, adhesives, etc. Alternately head portion 14 may be removably secured to handle portion 12 using any known fastening technique including threading, friction, set screw(s), etc. By providing rasp 10 with a removable head portion 14, different size head portions could be provided with a single handle portion such that the appropriate size rasp head portion could be chosen during a surgical procedure to accommodate a particular size intervertebral opening. Alternately, handle portion 12 and head portion 14 may be formed of monolithic construction (not shown). Preferably, rasp 10 is formed of a surgical grade stainless steel. However, other materials suitable for surgical use and meeting the requisite strength requirements could also be used to manufacture rasp 10 including plastics, ceramics, metals, etc.

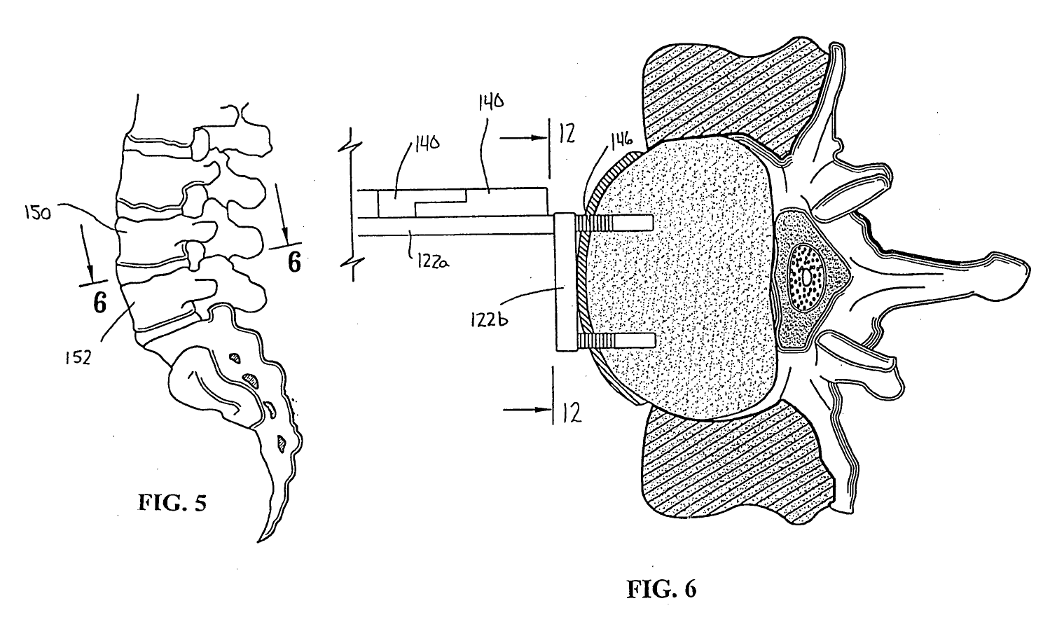

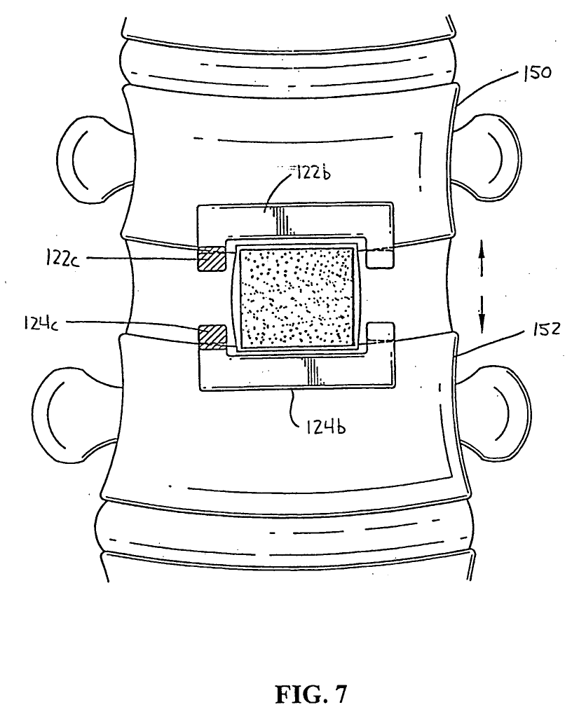

Vertical distractor 100 and rasp 10 can be used during an anterior or posterior surgical spinal fusion procedure adjacent vertebrae and to prepare the vertebrae endplates for receiving an implant. Additionally, vertical distractor 100 can be used for during anterolateral as well as transforaminal lumbar interbody fusion procedures. During a typical surgical spinal fusion procedure, the surgeon will perform a partial or total discectomy to prepare the intervertebral space for implant insertion. For example, the surgeon may perform a block discectomy, leaving the outer annulus of the intervertebral disc intact, while removing disc material to provide an anterior or posterior opening. Thereafter, the surgeon will use a trial or gauge to determine the size of the disc space and thus, the size of the implant required for the surgical procedure. A suitable trial is disclosed in U.S. patent application Publication Ser. No. 2002/0,016,633, the contents of which are incorporated by reference herein. A slaphammer may be used to remove the trial from the intervertebral space. Next, as illustrated in FIGS. 5-7, the surgeon will position arms 122c and 124c of jaws 122 and 124, respectively, into the intervertebral space such that grooved surfaces 146 engage the vertebral endplates. Operating members 112 and 114 of vertebral distractor 100 are compressed to distract the adjacent vertebrae 150 and 152 a desired amount. Locking member 132 can be adjusted to maintain the vertebrae at the desired degree of distraction. Because a pair of spaced arms are provided on each jaw, distractor 100 will provide a parallel distraction of vertebrae by orienting each jaw with the appropriate lordotic or kyphotic angulation. The trials and rasps may be similarly configured with surfacing to create a parallel, lordotic or kyphotic orientations. Thereafter, the surgeon will use the rasp to remove any remaining disc material from the vertebral endplate and to remove a portion of the cortical endplate to provide bleeding bone. Bleeding bone improves and quickens fusion of the implant and the vertebrae. After the vertebral endplates have been prepared using the rasp, the surgeon will again check the size of the intervertebral space with a trial. An implant can now be inserted into the intervertebral space between jaws 122 and 124. In an alternate embodiment, the surgeon may use the rasp on the vertebral endplates prior to distracting the vertebrae with the vertebral distractor 100.

It is to be understood that the above description is intended to cover all the generic and specific features of the apparatus and method described and all statements of the scope of the invention which, as a matter of language, might be said to fall therebetween. Moreover, the apparatus may be modified to include additional features not currently shown. For example, a gauge may be attached to the distractor to indicate the actual degree or distance of distraction. Therefore, the above description should not be construed as limiting, but merely as exemplifications of preferred embodiments. Thos skilled in the art will envision other modifications within the scope and spirit of the claims appended hereto.

What is claimed is:

1. A vertebral distractor which comprises:
a) a pair of operating members each of which possesses a proximal portion adapted for manual gripping and a distal portion adapted for spreading vertebrae, the operating members being connected to each other by a pivot member disposed between their proximal and distal portions;
b) a pair of extension members, each extension member being connected to the distal portion of each operating member and laterally offset on the same side thereof; and,
c) at least one vertebral endplate-engaging member connected to each extension member.

2. The distractor of claim 1 wherein the biasing action of a biasing member connected to the proximal portions of the operating members is counteracted by an adjustable locking member engagably connected to the operating members.

3. The distractor of claim 2 wherein the biasing member is a spring and the adjustable locking member is a screw and nut assembly.

4. The distractor of claim 1 wherein the distal portion of each operating member is slidably and pivotally connected to its proximal portion.

5. The distractor of claim 4 wherein the distal portions of the operating members are supported by a pair of rotatably connected support members.

6. The distractor of claim 5 wherein each support member possesses a proximal portion pivotally connected to the proximal portion of an opposing operating member, and a distal portion pivotally connected to the distal portion of the operating member.

7. The distractor of claim 1 wherein the distal portion of the distractor is at an angle relative to the proximal portion of the distractor.

8. The distractor of claim 1 wherein the end plate-engaging member of each extension member possesses a pair of spaced-apart arms having vertebral endplate-engaging surfaces.

9. The distractor of claim 8 wherein the vertebral endplate-engaging surfaces are contoured for improved gripping contact with the vertebral endplates.

10. The distractor of claim 8 wherein each arm terminates in a configuration which facilitates entry of the arms into the intervertebral space.

11. The distractor of claim 1 wherein the biasing action of a biasing member connected to the proximal portions of the operating members is counteracted by an adjustable locking member engagably connected to the operating members, the distal portion of each operating member is slidably and pivotably connected to its proximal portion with the distal portions of the operating members being supported by a pair of rotatably connected support members and the vertebral endplate-engaging member of each extension member possesses a pair of spaced-apart arms having vertebral endplate-engaging surfaces.

12. A rasp for the removal of tissue from a vertebral endplate which comprises an elongate handle portion terminating in a head portion having upper and lower surfaces with at least one such surface having a rasping configuration.

13. The rasp of claim 12 wherein the upper surface and/or lower surface of the head portion includes a multiplicity of rasping protrusions possessing an arcuate wall, a sloped wall and an edge.

14. An instrument set for carrying out an intervertebral fusion procedure which comprises the vertebral distractor of claim 1 and at least one of a rasp, trial, fusion implant inserter, slaphammer and bone tamp.

15. An instrument set for carrying out an intervertebral fusion procedure which comprises the vertebral distractor of claim 10 and at least one of a rasp, trial, fusion implant inserter, slaphammer and bone tamp.

16. The instrument set of claim 14 including the rasp of claim 12.

17. The instrument set of claim 15 including the rasp of claim 12.
